(19) United States
(12) Patent Application Publication
(10) Pub. No.: US 2007/0172425 A1
(43) Pub. Date: Jul. 26, 2007

(54) Testing cell cycle regulation effect of a compound using a hollow fibre cell implant

(76) Inventor: DongFang Liu, Waltham, MA (US)

Correspondence Address: ASTRAZENECA R&D BOSTON, 35 GATEHOUSE DRIVE, WALTHAM, MA, 02451-1215, US

(21) Appl. No.: 10/557,951

(22) PCT Filed: May 27, 2004
(86) PCT No.: PCT/GB04/02254

§ 371 Date: Oct. 4, 2006

Related U.S. Application Data
(60) Provisional application No. 60/474,552, filed on May 29, 2003.

(51) Int. Cl. A61K 49/00; G01N 33/574

(52) U.S. Cl. 424/9.2; 435/7.23

(57) ABSTRACT

The invention includes an in vivo pharmacodynamic method for testing a compound for cell cycle regulation.

### CROSS-REFERENCE TO RELATED APPLICATION

This application is related to U.S. Provisional Application 60/474,552, filed May 29, 2003, which is incorporated herein by reference in its entirety.

### FIELD OF THE INVENTION

The present invention relates to an in vivo pharmacodynamic (PD) method for testing a compound for cell cycle regulation. More particularly, the invention relates to an in vivo PD method for testing a compound for cell cycle checkpoint inhibition.

### INTRODUCTION

The tremendous advancement in cancer biology has revealed many potential molecular targets for therapeutic intervention. As most human cancers display deregulated cell cycle control (Morgan, D. O. Nature 374, 131-4 (1995)), the regulatory molecules associated with cell cycle control are proven to be valid cancer targets (Webster, K. R. Exp. Opin. Invest. Drugs 7, 865-887(1998); Webster, K. R. & Kimball, D. K. Emerging Drugs 5, 45-59 (2000)).

Cyclin-dependent kinases (CDKs) control the progression through the cell cycle, operating at the transition from the G2 to M and G1 to S phases, and progression through S. CDKs are regulated by a complex set of mechanisms, including the presence of activating cyclins, regulatory phosphorylations and checkpoint pathways (Webster, K. R. Exp. Opin. Invest. Drugs 7, 865-887(1998); Webster, K. R. & Kimball, D. K. Emerging Drugs 5, 45-59 (2000)); (Roy, K. K. & Sausville, E. A. Curr Pharm Des 7, 1669-87 (2001); Sausville, E. A. Ann N Y Acad Sci 910, 207-21; discussion 221-2 (2000)). The checkpoints at G1, S, G2, and M serve to monitor and ensure the integrity of genetic material before cells commit to DNA replication and mitosis. Upon activation, these checkpoint pathways interface with cyclin-Cdk complexes to halt the normal cell cycle of growth and division (Sampath, D. & Plunkett, W. Curr Opin Oncol 13, 484-90 (2001)). At the G2 checkpoint, the activation of cyclin B/CDK1 complex requires removal of the inhibitory phosphorylations on Thr-14 and Tyr-15 by the action of CDC25C phosphatase (Peng, C. Y. et al. Science 277, 1501-5 (1997); O'Connell, M. J. et al. Embo J 16, 545-54 (1997)). Upstream kinases Chk1 and 2, which are activated through phosphorylation by ATM and ATR upon DNA damage, negatively regulate CDC25C (Zhou, B. B. & Elledge, S. J. Nature 408, 433-9 (2000); Zhou, B. B. et al. J Biol Chem 275, 10342-8 (2000)). The inhibition of DNA damage-induced G2 checkpoint activation results in premature mitosis and cell death. Known G2 checkpoint inhibitors include caffeine, UCN-01, Go6976, SB-218078 and isogranulatimide (Jeffrey, R. et al. Cancer Res 60, 566-572 (2000); Roberge, M. et al. Cancer Res 58, 5701-5706 (1998)) which sensitize tumor cells to either radio or chemotherapy by preventing cells from arresting at the G2 checkpoint and repairing the DNA damage before entering mitosis (Zhou, B. B. et al. J Biol Chem 275, 10342-8 (2000); Graves, P. R. et al. J Biol Chem 275, 5600-5 (2000); Bunch, R. T. & Eastman, A. Clin Cancer Res 2, 791-7 (1996); Eun Kyung Choi, S. D. A. et al. Frontiers in Cancer Prevention Research 88 (Boston, 2002); Kohn, E. A. et al. J Biol Chem 277, 26553-64 (2002); Wang, Q. et al. J Natl Cancer Inst 88, 956-65 (1996); Yu, L. et al. J Biol Chem 273, 33455-64 (1998)).

The efficient development of target-based cancer therapeutics requires preclinical pharmacodynamic methods which enable clear in vivo demonstration of target inhibition and the associated change in functional or cell cycle endpoint (biological effects). However, a disadvantage of the majority of the existing assays and in vivo methods developed for testing traditional cytotoxics is that they are not sufficient or appropriate for the development of targeted cancer therapeutics. Furthermore, the primary end point of existing preclinical cancer methods is limited to physical measurement of the tumor size/growth or surrogate markers and provide little information as to whether a desired functional or cell cycle end point is achieved. For instance, in a conventional hollow fiber assay where mice carrying the fibers were repeatedly treated with a non specific CDK inhibitor, PCNA was used as a surrogate indirect marker of cell cycle regulation, which tells little about where and how the cells are arrested during cell cycles (Hall, L. A. et al. Anticancer Res 20, 903-11 (2000)). In addition, the existing methods using surrogate markers as the endpoints provide qualitative but not quantitative information; therefore, the existing methods are not suitable for compound-to-compound comparison purpose in the drug discovery cascade. In addition, existing methods apply unsynchronized cells with heterogeneous distribution of cell cycle profiles, which make the interpretation of the results difficult (Hall, L. A. et al. Anticancer Res 20, 903-11 (2000); Suggit, M. et al. European Journal of Cancer 38, 39 (2002)).

The hollow fibre assay was originally developed by Hollingshead et al. as an additional in vivo efficacy method for screening and identifying compounds with potential anti-cancer activities, and the information thus derived was used as a prioritization tool for further testing in the xenograft model (Plowman, J. D et al. Hollow Fibre Assay: A new approach to in vivo drug testing, 119-121 (Humana Press, Totowa, N.J., 1997); Casciari, J. J. et al. J Natl Cancer Inst 86, 1846-52 (1994); Hollingshead, M. G. et al. Life Sci 57, 131-41 (1995)). Survival of the cancer cells is used as the endpoint, which is typically measured by means of an MTT (3-[4,5-dimethylthiazol-2-yl]-2,5-diphenyltetrazolium bromide) dye conversion assay (Hollingshead, M. G. et al. Life Sci 57, 131-41 (1995)). Attempts were made to compare protein level change as an indirect indicator of cell cycle progression by carrying out Western analysis on hollow fibre derived cells (Hall, L. A. et al. Anticancer Res 20, 903-11 (2000). This technique has limited use as a pharmacodynamic method in studying the mechanism of drug actions. Furthermore, the existing standard hollow fibre assay typically takes about 7-10 days and involves multiple compound dosings for the reason that multiple doublings of cells are required for a compound to exhibit anti-cancer activities with the existing hollow fibre assay (Plowman, J. D. et al. Hollow Fibre Assay: A new approach to in vivo drug testing, 119-121 (Humana Press, Totowa, N.J., 1997); Hall, L. A. et al. Anticancer Res 20, 903-11 (2000); Hollingshead, M. G. et al. In vivo cultivation of tumor cells in hollow fibres. Life Sci 57, 131-41 (1995)).

### SUMMARY OF THE INVENTION

The present invention provides, in part, a method for studying cell cycle regulation, in particular for screening of compounds that target specific components of the cell cycle. The invention also provides an in vivo pharmacodynamic method that can be used to study the mechanism of action and the pharmacodynamic-pharmacokinetic (PK)-efficacy relationship of compounds with rapid throughput. Moreover, the present method can further include determining the toxicity of a drug of interest.

An advantage of the present invention is that it provides an in vivo pharmacodynamic method which greatly reduces the amount of time spent in conducting a typical study and the accompanying materials and animal usage.

Accordingly, the invention includes an in vivo pharmacodynamic method for testing a compound for cell cycle regulation. The method includes:
i) implanting a semi-permeable cell receptacle comprising a cell into an animal;
ii) administering a test compound to said animal in vivo; and
iii) determining a cell cycle endpoint in the cell, whereby a progression or arrest of a cell cycle phase in the cell indicates that the compound is a cell cycle regulator. The progression of arrest of the cell includes release from arrest at a cell cycle phase of previously arrested cells or prevention of arrest at a cell cycle phase, for example, of subsequently arrested cells, or both. A particular example of cell cycle regulation is cell cycle checkpoint inhibition and a particular example of a cell cycle regulator is a cell cycle checkpoint inhibitor.

A semi-permeable cell receptacle includes a sealable cell receptacle comprising a semi-permeable membrane permitting transmembrane exchange of molecules but not cells. Preferably, the semi-permeable cell receptacle is a hollow fibre.

An arrested cell includes an arrested cell or synchronized cell.

The invention is also suitable for testing one or a number of compounds, or can be used in a compound screening cascade in drug discovery operations.

A cell cycle regulator includes a test compound which is capable of releasing from arrest at a cell cycle phase of a previously arrested cell, preventing from arrest at a cell cycle phase, for example, of a subsequently arrested cell, or both.

A “cell cycle phase” can be any of the traditional subdivisions of the standard cell cycle, that is the G1, G2, S or M phase.

It will be appreciated that there are numerous means to carry out the above method such that a functional or cell cycle endpoint may be determined. In a first example, the cells may be arrested prior to or after loading the cell receptacle. When cells are arrested prior to or after loading the cell receptacle, the cell receptacle may then be implanted into the animal prior to or after administering the compound to the animal. It can be envisaged that the cell receptacle may be implanted into the animal after compound administration to the animal where the compound to be tested may be a long-acting compound. A functional or cell cycle endpoint may then be measured. The cell receptacles can be implanted in any appropriate place in the body, for example, subcutaneously, intraperitoneally, or a combination of both.

In a second example, the cell receptacle may be loaded with cells and may then be implanted into the animal. The cells are then arrested prior to or after administering the compound to the animal. A cell cycle endpoint may then be measured, i.e., cell cycle progression or arrest.

In a third example, the cell receptacle may be loaded with cells and compound may be administered to the animal. The cell receptacle may then be implanted into the compound-administered animal prior to or after arresting the cells. A cell cycle endpoint may then be measured.

Methods known in the art can be used to measure cell cycle endpoint such as FACS analysis.

In one embodiment, the cells are arrested at a particular cell cycle phase, for example, at the G1 phase. By the term “G1” or “G1 phase”, we mean the phase of the cell cycle between the completion of mitosis and the beginning of DNA synthesis.

In another embodiment, the cells are arrested at the S phase. By the term “S” or “S phase”, we mean the phase of the cell cycle when DNA is replicated.

In yet another embodiment, the cells are arrested at the G2 phase. By the term “G2” or “G2 phase”, we mean the phase of the cell cycle between the end of DNA synthesis and the beginning of mitosis.

In another embodiment, the cells are arrested at the M phase. By the term “M” or “M phase”, we mean the phase of the cell cycle when chromosomes are separated into two daughter cells.

In a typical study incorporating the invention, the cells may be arrested at the G1 phase, the S phase, the G2 phase, and/or the M phase. Preferably, the cells are arrested at the G1 and/or G2 phase. Arresting the cells at the G1 phase, the S phase, the G2 phase and/or the M phase enables the study of targets in the regulatory pathways that interface with these cell cycle phases (Senderowicz, A. M. & Sausville, E. A. J Natl Cancer Inst 92, 376-87 (2000); Sausville, E. A., Johnson, J., Alley, M., Zaharevitz, D. & Senderowicz, A. M. Ann N Y Acad Sci 910, 207-21; discussion 221-2 (2000). Examples of such targets include CDKs 1, 2, 4 and 6; cyclins A, B, D and E; Cdc25C; Cdc25A; Chk1/2; Wee 1; Myt 1; ATM; ATR; TAK; CAK; p53, p21; p27; RB; tublin; kinesin related motor proteins such as HsEg5; aurora kinase.

Preferably, the cells are arrested at the G1 phase, the S phase, the G2 phase, and/or the M phase by administering a DNA damaging agent. A “DNA damaging agent” includes any substance or treatment, which induces DNA damage, causes cell cycle arrest or induces cell cycle synchronization in a cell, including Gamma irradiation, UV irradiation, X-rays, alkylating agents, antibiotics that induce DNA damage, inhibitors of topoisomerase. Examples of specific compounds are cisplatin, carboplatin, thioptepa, carmustine, cyclophosphamide, temozolomide, ifosfamide, Carmustine (BCNU), melphalan, topotecan, irinotecan, SN-38, camptothecin, VM-26 (teniposide), etoposide, actinomycin D, bleomycin, mitomycin, anthracyclines, and doxorubicin.

Preferably, the cells are arrested at the G1 phase, the S phase, the G2 phase, and/or the M phase by administering an antimetabolite. “Antimetabolite” include any substance or treatment that bearing a structural resemblance to one required for normal physiological functioning, exerting its effect by interfering with the utilization of the essential metabolite, and leading to cell cycle arrest or synchronization in a cell. Examples of specific compounds are methotrexate, 5-fluorauracil, mimosine, hydroxyurea, aphidicolin, cytarabine, gemcitabine, mercaptopurine, and other inhibitors of DNA synthesis. Some antimetabolites may also indirectly cause DNA damage.

Preferably, the cells are arrested at the G1 phase, the S phase, the G2 phase, and/or the M phase by administering an antiproliferative. “Antiproliferative” include any substance or treatment that interferes with normal cellular functions that leads to cell cycle arrest or synchronization in a cell. Examples of antiproliferative substances and treatment are thymidine treatment, serum starvation, nocodazole, vinblastin, paclitaxel, docetaxel, cyclosporin A, rapamycin, cycloheximide, tamoxifen, anastrozole, imatinib, Gefitinib (IRESSA) and any other compounds that block cell cycle progression by interfering with signaling pathways that regulate cell cycle progression. Particularly preferred antiproliferatives include thymidine treatment.

In another aspect, the invention further comprises the step of determining whether a protein associated with the cell cycle phase is affected by the compound. The protein may be associated with the G1 phase, the S phase, the G2 phase, or the M phase and their associated checkpoint pathways. Preferably, the proteins are associated with the G2 phase, the proteins being selected from the group consisting of ATR, ATM, Chk 1, Chk2, CDK1 (CDC2), Myt 1, Wee 1, Cdc25C, Cdc25A and cyclin B. Particularly preferred proteins are Chk1, cdc25c and CDK1 (CDC2).

In yet another aspect, determining whether the protein associated with the cell cycle phase is affected by the compound comprises:
i) lysing the cells to produce a cell extract containing proteins from the cell;
ii) separating proteins from the cell extract to produce a profile of proteins; and
iii) comparing the profile to a profile obtained from cells not so treated with the compound.

Preferably, Western blot analysis is used to produce the profiles that are compared. The data may be further analysed. For example, commercially available hardware may be used which allows for measurement of band intensity. Further analytical techniques can be envisaged, such as protein array analysis, mass spectrometry based method, HPLC, immunofluorescent detection, and multiplex protein analysis technologies (e.g. Bioplex/luminex). In addition, cells harvested from the hollow fibres can be used for immunohistochemistry study after cytospin on a glass slide.

In another aspect, the invention further comprises the step of determining whether DNA or RNA associated with the cell cycle phase is affected by the compound. The DNA or RNA may be associated with the G1 phase, the S phase, the G2 phase or the M phase and their associated checkpoint pathways.

In yet another aspect, determining whether the DNA or RNA associated with the cell cycle phase is affected by the compound comprises the steps of:
i) lysing the cells to produce a cell extract containing DNA or RNA from the cell;
ii) separating the DNA or RNA from the cell extract to produce a profile of DNA or RNA, and
iii) comparing the profile to a profile obtained from cells not so treated with the compound.

Alternatively, DNA or RNA can be extracted from the cell lysates and analyzed for genetic or epigenetic changes as a result of compound treatment with techniques including micro-array, PCR, RT-PCR, real-time PCR, in situ hybridization, sequencing, Northern blot and Southern blot. Examples of epigenetic changes include DNA methylation, imprinting, histone acetylation and methylation, enhanced or decreased promoter activity. Examples of genetic changes include the up or down-regulation of gene expression, mutation, loss of heterogeneity (LOH), cytogenetic alterations and chromosome abnormalities.

Preferably, the cells are capable of arrest at any of the G1, S, G2 or M phases.

The cells used in the present invention can be any cell such as a primary cell, secondary cell, or an immortalized cell. In one embodiment, the cell is a peripheral blood mononuclear cell (PBMC). The cell is preferably a tumour cell. Genetically engineered cell lines may be used to generate a desired cell cycle arrest profiles. For instance, p53 knock out in tumor cells result in primarily cell cycle arrest at G2 checkpoint.

Cells may be engineered with a reporter gene/marker construct containing either a single reporter gene (transgene) or combined reporter genes (e.g. fusion protein, or cis-linked reporter gene, or multiple reporter vectors) such as but not limited to luciferase, renilla luciferase, β-galactosidase, green, red, yellow, blue fluorescent protein, thymidine kinase. Specific reporter probe(s) such as radiolabeled probes (e.g., *I-FIAU, or ¹⁸F-FHBG) may be used to monitor the level of transgene expression (e.g., HSV1-TK). Activity of the reporter gene in the cell receptacle after treatment may be monitored in cell lysates or non-invasively in vivo without retrieving the cell receptacle loaded with cells from the animal with molecular imaging technique such as optical imaging, or nuclear imaging such as PET (positron emission tomography), microPET, or GAMA camera The reporter gene may be either expressed constitutively or inducibly under the control of a promoter/enhancer, where the level of reporter gene expression serves as a sensor for the promotor activity. Preferably, the expression of the reporter gene is under the control of cell cycle specific promoters such as E2F-1 promoter controlled expression of luciferase as a sensor for cell cycle progression.

Preferably, the cells are tumour cell lines. Such tumour cell lines include cell lines included in NCI-60 cell panel, B16 melanoma cells, Lewis lung carcinoma and any cell lines that are derived from these cell lines such as p53 Knockout and dominant negative cell lines. Particularly preferred tumour cell lines include HCT116, HT29 and H460 cells. The HCT116 cells may be wild type or genetically engineered cells. The HT29 cells may be wild type or genetically engineered cells. The H460 cells may be wild type or genetically engineered cells such as p53 dominant negative.

In a further aspect, administering the test compound comprises injecting the animal with the compound. The compound can be administered systemically, for example, the compound can be administered intraperitoneally, intravenously, intramuscularly, orally, pulmonarily (pulmonary inhalation/delivery), or delivering with a slow release device such as an osmotic pump or polymer based slow-release formulation. Further administration routes may also be envisaged, the administration route chosen being dependent upon the pharmacokinetic and physical properties of the compound.

Examples of an animal that can be used in the invention include a rodent, rabbit, dog, rhesus monkey, and chimpanzee. Preferably the animal is a mammal such as a human. Particularly preferred rodents include a rat and a mouse. Immunocompromised rodents, for example nude rats and mice are preferred for direct pharmacodynamics-efficacy correlation studies.

In a further aspect, the invention comprises use of a method as defined hereinabove for performing a pharmacokinetic-pharmacodynamic-efficacy correlation study.

Preferably, the invention further comprises determining an optimal biological dose and/or dosing schedule for an in vivo efficacy study. The optimum biological dose may be used in a clinical trial design protocol, and particularly in a human clinical trial design protocol.

One advantage of the present invention is that it provides the first validated in vivo pharmacodynamic method for study of cell cycle regulation with quantifiable functional or cell cycle endpoints. For example, this pharmacodynamic method allows direct, unambiguous in vivo mechanistic study of the drugs designed to modulate the checkpoint pathways.

Another advantage of the invention is it enables examination of molecular markers as indicators of target inhibition and associated change in functional or cell cycle end-points or biological effects in addition to the general, nonspecific endpoints such as cell death and proliferation.

Additionally, the invention allows for pharmacokinetic-pharmacodynamic-efficacy correlation studies and the determination of the optimal biological dose for targeted therapeutics that could prove valuable for both drug discovery and clinical developments. Also, parameters can be developed allowing judgment of the quality of the experiment as well as comparison of the potency and pharmacokinetics of compounds across different experiments.

The invention enables rapid in vivo screening of test compounds targeting specific components of the cell cycle checkpoint pathways in development of cell cycle targeted therapeutics.

One additional advantage of the present invention is that in contrast to that of xenograft experiments, a typical study requires minimal use of animals, hours to a few days after fiber implantation. For example, as few as one mouse can give a readout using FACS analysis. Also, minimal use of compounds, for example as little as 1 to 10 mg of single or multiple doses, are required. Single doses are preferable. These features of the pharmacodynamic model also compare favorably with that of a standard conventional hollow fiber assay, which typically takes 7-10 days and requires multiple compound dosings.

### BRIEF DESCRIPTION OF THE DRAWINGS

The invention will now be described, by way of example only, with reference to the accompanying drawings in which:

FIGS. 1A-H depict graphs showing results from an in vitro topotecans (TOP) titration with HCT116 (A, B, C, D) and H460 (E, F, G, H) cells.

FIGS. 2A-I depict graphs showing results from an in vitro study with H460 (A, B, C, D, E) and HCT116 (F, G, H, I) cells. After topotecan treatment, the G2 arrested cells were incubated with fresh medium in the presence of Go6976 (D, H), caffeine (E, G), and compound X (I) for a total of 24 hours.

FIG. 3A depicts a scheme of the experimental procedure for the in vivo study with caffeine and compound X using G2 arrested HCT116 cells in hollow fibres. FIGS. 3B-H depict graphs showing results from an in vivo study with caffeine and compound X using G2 arrested HCT116 cells in hollow fibres.

FIGS. 4A-F depict graphs showing results from an in vivo study with compound Y using G2 Topotecan arrested HCT116 cells in hollow fibres. In this study, hollow fibres are harvested 30 hours after implantation and compound treatment.

FIG. 5A is a histogram showing phosphor/total cdc25c ratio. FIG. 5B is a histogram showing phosphor/total cdc2 ratio.

FIG. 6A-J depict graphs showing in vivo Gamma Irradiation titration with hollow fibres. FIG. 6K-P depict graphs showing dose- and schedule-dependent G2 checkpoint inhibition with H460, p53 DN cells by compound Y.

FIGS. 7A-C depict graphs showing in vitro synchronization of HCT116 cells with double thymidine block (DTB). FIGS. 7D-F depict graphs showing induction of G2 arrest in synchronized HCT116 cells with brief topotecan (TOP) treatment.

FIG. 8A-F depict graphs showing double thymidine block with HCT116 cells in hollow fibre and in vivo cell cycle progression after implantation.

FIG. 9A-B depict histograms showing results of a simultaneous determination of PD activity and bone marrow toxicity for compound Y in mice.

FIG. 10A-D depict graphs showing a simultaneous determination of plasma PK, hollow fibre PK, PD activity in hollow fibre, and bone marrow toxicity for compound Y in immune competent rats.

FIG. 11 depicts a schematic which demonstrates the potential applications of the pharmacodynamic (PD) method in drug discovery.

### DETAILED DESCRIPTION OF THE INVENTION

The invention enables rapid in vivo screening of test compounds targeting specific components of the cell cycle checkpoint pathways in development of cell cycle targeted therapeutics. The invention involves loading a semi-permeable cell receptacle such as a hollow fibre with cells and subsequently implanting the hollow fibre into an animal. Cells are treated with an agent such as a DNA damaging agent, for example, either prior to or after implantation under conditions whereby arrest at a cell cycle phase will be induced. A compound to be tested for cell cycle regulation, for example checkpoint inhibition activity, is administered to the animal so as to affect the cells that are targeted, following which it is determined whether the compound is a cell cycle checkpoint regulator, for example, a cell cycle checkpoint inhibitor. Release of the cells from arrest or prevention of arrest at the cell cycle phase, such that the cells proceed to mitosis and G1, for example, is the indicator of G2 checkpoint regulation. In another embodiment, the cells are synchronized.

Once the conditions for arrest of the cells at a cell cycle, or conditions whereby the cells are synchronized, are established, administration of the compound to be tested may be carried out. For example, the cells may be arrested at a cell cycle phase and then treated with the test compound to determine whether there is release from arrest at the cell cycle phase. Alternatively, the cells or the animals carrying the hollow fibres may be treated with the test compound prior to arrest of the cells at the cell cycle phase and determine whether the cells are prevented from arrest at the cell cycle phase.

Release from arrest at the cell cycle phase or prevention of arrest at the cell cycle phase is detected by a quantitative determination of the cells at a particular phase of the cell cycle. For example, release from G2 arrest or prevention of G2 arrest is detected by a quantitative determination of the cells which proceed to mitosis and reenter G1 or undergo cell death. This approach is facilitated by the use of FACS analysis which, by using DNA measurements, allow large numbers of cells to be analysed automatically.

Conditions for arresting the cells at a cell cycle phase in response to a DNA damaging agent, for example, may be optimized by determining appropriate incubation time, concentration/dose, and type of the DNA damaging agent. Maximising the proportion of cells in the population which are arrested at a cell cycle phase will reduce the background signal. For example, preferably, at least 30% of the cells in a hollow fibre will be arrested at G2 in response to the DNA damaging agent.

Compounds tested positively in the assay will result in the identification of those compounds having not only checkpoint inhibition activity, but also reasonable in vivo PK properties. For example, compounds X or Y have been shown in this study to be a G2 checkpoint inhibitor in vivo.

Cell cycle checkpoint inhibitors may be used in the treatment of cancer or other proliferative diseases.

In a particular embodiment of the invention, tumor cells loaded into hollow fibres were arrested at the G2 phase by means of either Topotecan or gamma irradiation either before or after implantation to mice, which subsequently was used to study the regulation pathways associated with the cell cycle arrest. The model was validated both in vitro and in vivo with known regulators of cell cycle; for instance, systemic injection of caffeine, compound X and compound Y to mice was shown to inhibit topotecan induced G2 arrest in tumor cells in vivo. The check point inhibition is observed as a reduction in the percentage of cells in G2 (% G2) and/or increase in % G1.

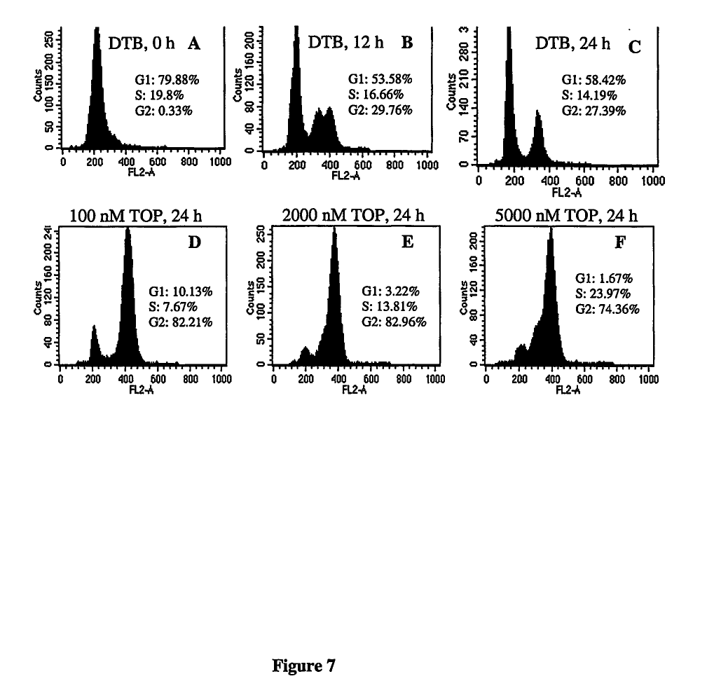

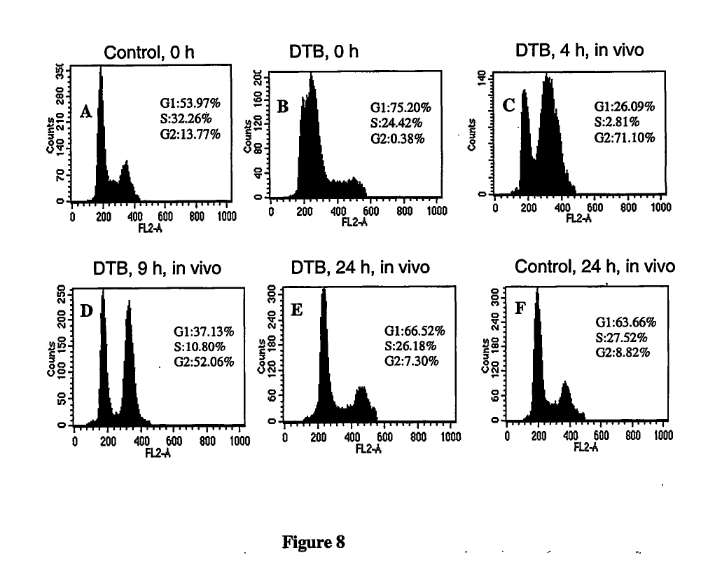

In another embodiment of the invention, cells are synchronized at the G1/S boundary and then released to progress through the cell cycle both in vitro and in vivo (FIGS. 7, 8). Synchronizing cells at the G1/S boundary allows detection of either inhibition or acceleration of cell cycle progression through S and G2/M phases and thus the study of targets involved in S, and M phase progression regulation, such as CDKs 2, 4 and associated regulatory pathways. It is conceivable that CDK inhibitors would arrest cells at the corresponding cell cycle phase, while inhibitors of S and G2 checkpoint would accelerate the progression of cell cycle after DNA damage (Kohn, E. A. et al. J Biol Chem 277, 26553-64 (2002); Yu, L. et al. J Biol Chem 273, 33455-64 (1998)). For instance, UCN01 was described in literature to be able to transit cells directly from S phase to M phase followed by apoptosis (Bunch, R. T. & Eastman, A. Clin Cancer Res 2, 791-7 (1996); Wang, Q. et al. J Natl Cancer Inst 88, 956-65 (1996); Yu, L. et al. J Biol Chem 273, 33455-64 (1998). When synchronization was combined with topotecan treatment, the cells were arrested by activating a particular checkpoint pathway, which is the G2 checkpoint in the examples described. This particular embodiment is advantageous in that all cells in the cell cycle are synchronized and proceed through the cell cycle in a homogenous fashion, therefore there is no confusion in interpreting the results and often there is less background. For example, topotecan treatment was targeted to a homogenous early S phase cell population, and there was very low or no G1 population. Therefore, any subsequent G1 population observed is solely from cells exiting G2 and M phases.

The invention also enables examination of molecular markers as indicators of target inhibition. Proteins involved in a cell cycle checkpoint pathway that may be affected by a compound, are identified by their difference in cells treated with the compound as compared to cells not so treated. Proteins may be analysed by Western blotting, intensity measurements, and other known proteomics techniques such as mass spectrometry, multiplex protein analysis, protein array analysis, HPLC, immunofluorescent detection, etc.

Alternatively, cells engineered with a reporter gene/marker construct containing either a single reporter gene (transgene) or combined reporter genes (e.g. fusion protein, or cis-linked reporter gene, or multiple reporter vectors) such as but not limited to luciferase, renilla luciferase, β-galactosidase, green, red, yellow, blue fluorescent protein, thymidine kinase can be loaded into the hollow fibre. Specific reporter probe(s) such as radiolabeled probes (e.g., *I-FIAU, or ¹⁸F-FHBG) can be used to monitor the level of transgene expression (e.g., HSV1-TK). Activity of the reporter gene in the hollow fibre after treatment can be monitored in cell lysates or in vivo with molecular imaging technique such as bioluminescent imaging, optical imaging, or nuclear imaging such as PET (positron emission tomography), microPET, or GAMA camera. The reporter gene can be either expressed constitutively or inducibly under the control of a promoter/enhancer, where the level of reporter gene expressed serves as a sensor for the promotor activity. Preferably, the expression of the reporter gene is under the control of cell cycle specific promoters such as E2F-1 promoter controlled expression of luciferase as a sensor for cell cycle progression.

Additionally, the invention enables examination of both molecular markers as indicators of target inhibition and associated change in functional or cell cycle end-points. For example, this in vivo pharmacodynamic model demonstrates both target inhibition (phosphorylation of CDC25C by CHK1) and associated biological effect (G2 checkpoint inhibition) after systemic delivery of a chk1 inhibitor. It is expected that other components of G2 checkpoint pathway, including ATR, ATM, chk2, CDK1, Myt 1, Wee 1, cdc25c and cyclin B, can be similarly examined.

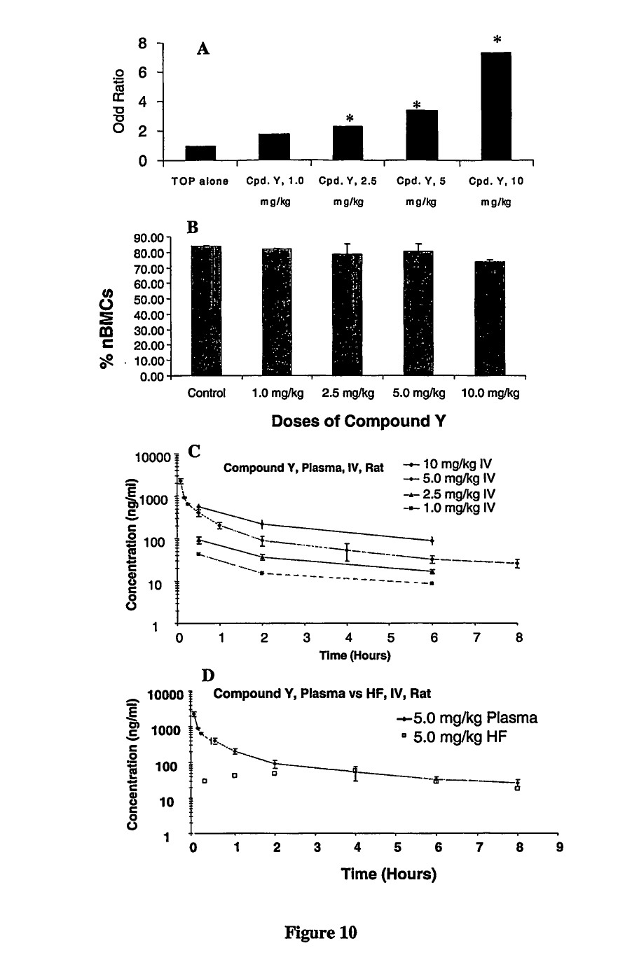

Specifically, the invention is amenable to dose-response relationship study where the doses of the compound are correlated to the extent of the checkpoint inhibition. By administrating compound at multiple dose levels, the invention is able to identify the minimum effective dose/concentration (Dₘᵢₙ/Cₘᵢₙ, the minimum dose/concentration showing checkpoint abrogation activity). In addition, exposure time and abrogation activity relationship can be established in this model by performing a dose-response relationship study where the plasma concentrations above in vitro cell EC50 (exposure time above EC50) can be directly correlated to the observed PD activities at the corresponding doses (FIG. 10). This information, when combined with the pharmacokinetic properties of a compound, is expected to guide dose and frequency selection for efficacy study. Therefore, the invention is expected to serve as the prioritizing tool for further efficacy testing, and provide a platform in the drug discovery cascade linking both pharmacokinetic and efficacy studies.

### EXAMPLES

### Example 1

Topotecan Induced G2 Arrest in vitro

To use inhibition of G2 arrest as a functional or cell cycle endpoint, it was necessary to induce a well-defined and relatively uniform G2 arrest in cells. Firstly, topotecan's concentration was titrated to identify the minimal dose requirement that leads to G2 specific arrest as a higher dose of topotecan could also result in S phase arrest (Kohn, E. A., Ruth, N. D., Brown, M. K., Livingstone, M. & Eastman, A. Abrogation of the S phase DNA damage checkpoint results in S phase progression or premature mitosis depending on the concentration of 7-hydroxystaurosporine and the kinetics of Cdc25C activation. J Biol Chem 277, 26553-64 (2002)). Topotecan was chosen primarily for two reasons; first, it is a standard agent broadly used in colon and lung cancer therapy; second, topotecan is an S phase specific agent where its inhibition of topoisomerase I results in DNA strand breaks. This is important because S-phase specific damage is not expected to activate G1 arrest mediated by p53, which has been documented in the literature (Kohn, E. A., Ruth, N. D., Brown, M. K., Livingstone, M. & Eastman, A. Abrogation of the S phase DNA damage checkpoint results in S phase progression or premature mitosis depending on the concentration of 7-hydroxystaurosporine and the kinetics of Cdc25C activation. J Biol Chem 277, 26553-64 (2002)).

To perform the experiments, cells were grown to 50% confluency in 10 cm dishes. The cells were then dosed with topotecan at indicated concentrations for 18 hours. Cells were then harvested and the G2 arrest was analyzed by FACS analysis 24 h later.

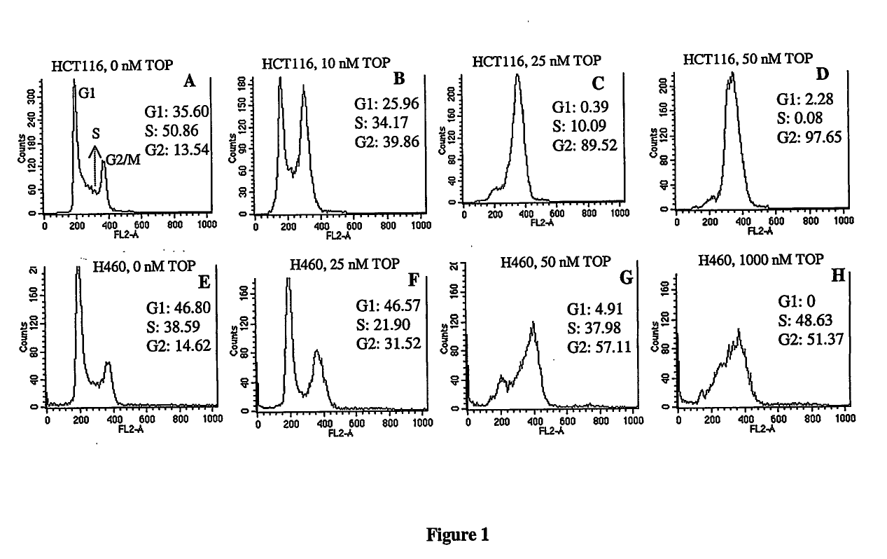

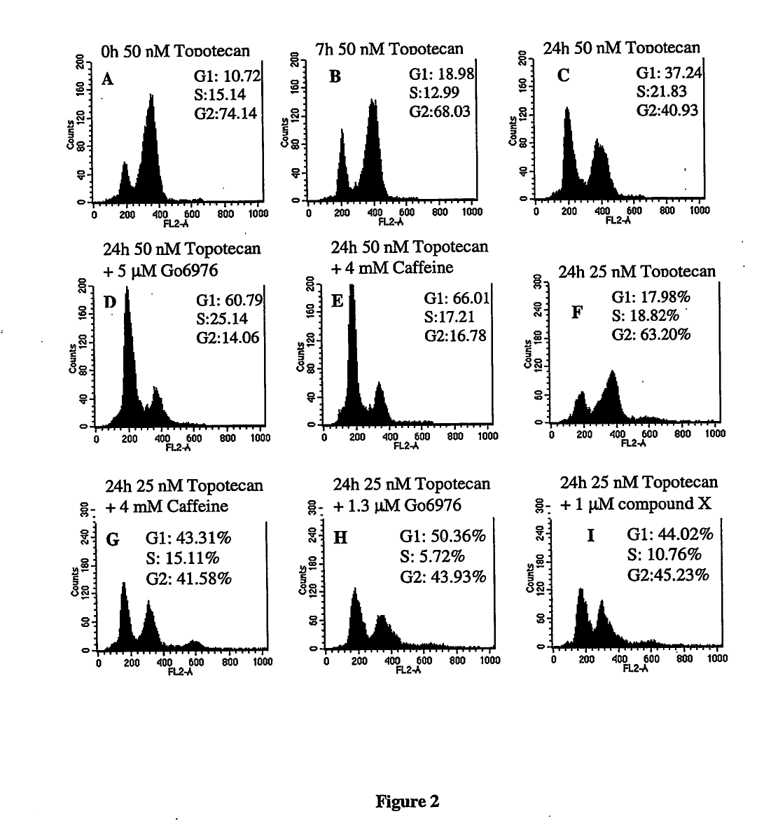

The results showed that both H460 and HCT116 cells are arrested either at the G2 or S phase checkpoint in a dose dependent manner (FIG. 1A-H). For HCT116 cells, 25 nM topotecan was shown to be the minimal concentration required to achieve an efficient G2 arrest while a higher concentration of topotecan begins to arrest cells at S phase as well (FIGS. 1A, B, C, D). For H460 cells, 50 nM sufficiently arrests cells at the G2 checkpoint while higher concentration arrest cells at both the S and G2 checkpoints (FIGS. 1E, F, G, H). As expected, no significant G1 arrest is observed. Importantly, we showed that both cell lines begin to reenter the cell cycle at about 7 h (H460) and 9 h (HCT116, not shown) and a noticeable percentage of cells leave G2 arrest and appear in G1 24 h after topotecan treatment (FIGS. 2A, B, C, F). Therefore, 24 h time point was chosen to compare treatment vs. control group in the subsequent checkpoint abrogation/inhibition assays. However, additional time points such as 30 h may also be used as the experimental endpoint.

Go6976, Compound X and Caffeine Inhibit Topotecan Induced G2 Arrest in vitro

Go6976, compound X and caffeine were then applied as known G2 checkpoint inhibitors in vitro, and were expected to accelerate the rate at which the arrested cells exit G2. The compounds were added to the cells after G2 arrest induced by 18 h topotecan treatment at the indicated concentrations and incubated for a period of 24 h. Cells were then harvested and analyzed by FACS. 2A, B, C and F are controls without abrogation treatment analyzed at indicated time points.

The results showed that Go6976 effectively inhibits G2 arrest at concentrations of 1.3 and 5 μM (FIGS. 2H, D). At this concentration, Go6976 alone does not show any signs of toxicity to cells as observed under microscope as well as by FACS analysis. Caffeine at 4 mM significantly inhibits topotecan induced G2 arrest in HCT116 and H460 cells (FIGS. 2E, G). Compound X effectively inhibits G2 arrest in HCT116 cells at 1 μM (FIG. 2I) without causing noticeable toxicity. It is noted that both Go6976 and compound X are significantly more potent G2 checkpoint inhibitors than caffeine (1 μM vs 4 mM).

### Example 2

Cells in Hollow Fibre Recapitulates in vitro Observations

The microenvironment for cells in the hollow fibre is different from the cells in culture dishes (Casciari, J. J. et al. J Natl Cancer Inst 86, 1846-52 (1994); Hollingshead, M. G. et al. Life Sci 57, 131-41 (1995)). Therefore, it was necessary to investigate whether the cells loaded into hollow fibres can recapitulate the in vitro observations in terms of the profiles in G2 arrest and checkpoint inhibition.

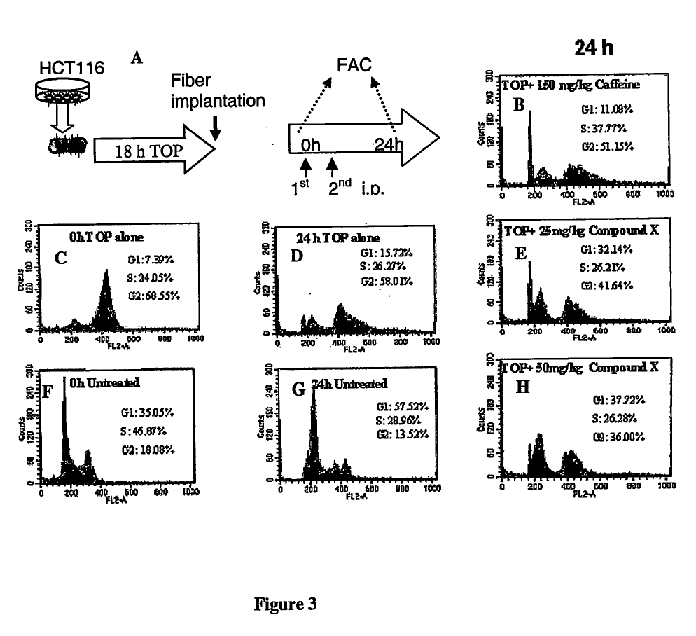

Specifically, HCT116 cells were first arrested at G2 with topotecan (TOP) treatment. At the end of 18 h topotecan treatment, fibres were implanted subcutaneously to mice with 4 fibres per mouse (FIG. 3A). Control fibres without topotecan treatment were also included (FIGS. 3F, G). Mice in the treatment group (n=2) received two sequential intraperitoneal (i.p.) caffeine (150 mg/kg/dose) (FIG. 3B) or compound X (FIGS. 3E, 25 mg/kg/dose; 3H, 50 mg/kg/dose) injections. The first injection was administered immediately after fibre implantation while the second injection was given 5 hours later. Mice in the TOP alone control group (n=2) received vehicle (0.9% NaCl) only (FIG. 3D). After implantation, fibres (8 fibres per time point) were retrieved from mice at indicated time points. Cells were then flushed out from the fibres and analyzed by FACS and immunoblotting.

The results from hollow fiber studies are consistent with the observations made in the cell culture experiments FIGS. 1, 3C, D, F, G). After subcutaneous implantation into nude mice, these arrested cells in hollow fibre exit G2 arrest at a similar rate to that of in vitro cell culture experiments (FIGS. 3C, D).

Caffeine and Compound X Inhibits G2 Arrest in vivo

We demonstrated G2 checkpoint inhibition with G2 checkpoint inhibitors such as caffeine and compound X. Caffeine has been shown in the literature to sensitize tumor to radiotherapy by inhibiting radiation induced G2 arrest at 100 and 200 mg/kg doses after intraperitoneal injection (Eun Kyung Choi, S. D. A., Yun-Hee Rhee, Hyun Sook Chung, Sung Whan Ha, Chang W. Song, Robert J. Griffin and HeonJoo Park. Frontiers in Cancer Prevention Research 88 (Boston, 2002)). Our results showed that the maximum tolerated dose (MTD) for caffeine in nude mice is 150 mg/kg, which was subsequently used for our in vivo study. Compound X is a selective chk1 kinase inhibitor and inhibits G2 arrest at 1 μM in vitro (FIG. 2I).

Caffeine inhibits/abrogates G2 arrest in two separate experiments with ΔG2% values (the difference in % G2 between the treatment and control groups) of 7% and 20%, respectively (Table 1A). This variation may be explained by the facts that the caffeine dose applied here is very high at MTD dose and caffeine is a relatively weak G2 checkpoint inhibitor.

Unlike caffeine, compound X exhibits more pronounced G2 checkpoint inhibition at both 25 and 50 mg/kg doses and the G2 checkpoint inhibition is dose dependent (17% and 22% in ΔG2% values, respectively) (FIGS. 3E, H; Table 1A).

Compound Y Inhibits G2 Arrest in vivo

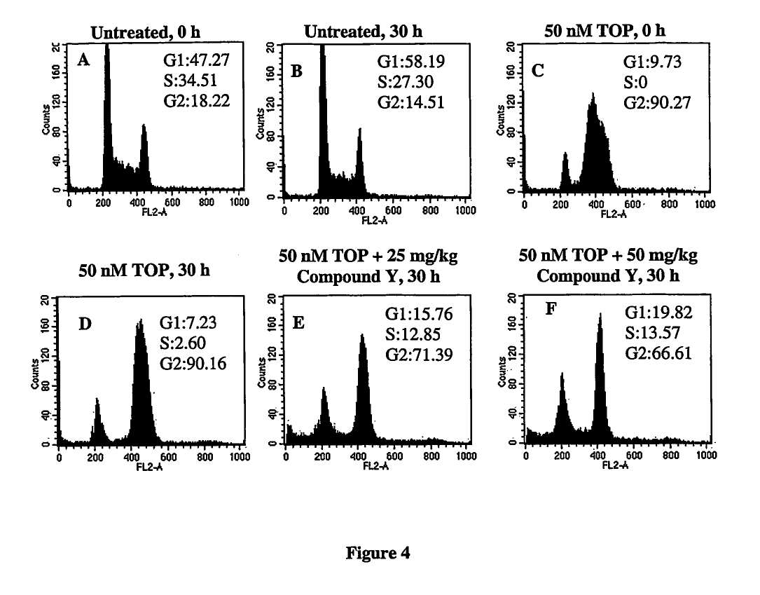

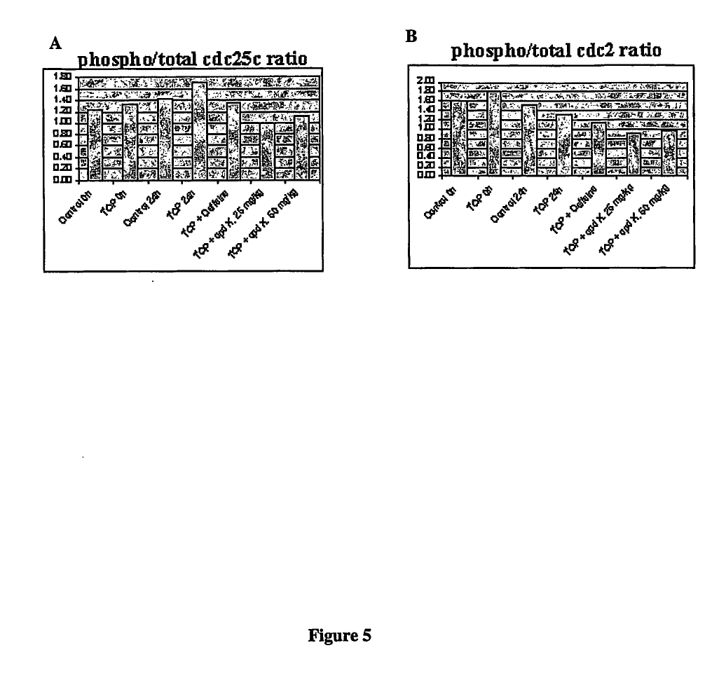

An in vivo study with compound Y using G2 Topotecan arrested HCT116 cells in hollow fibres was also conducted as follows. HCT116 cells in the hollow fibers were arrested at G2 by treating with 50 nM topotecan for 18 h and implanted immediately after (time 0 h). About 90% of cells were arrested at G2/M phase at time 0 h (FIG. 4C) and the arrest persisted until time 30 h (FIG. 4D). Compound Y or vehicle was administered at the indicated doses with a single intravenous injection shortly after the implantation (FIGS. 4E, F). Cells that did not receive any compound treatment were harvested at time 0 and 30 h as controls (FIG. 4A, B).

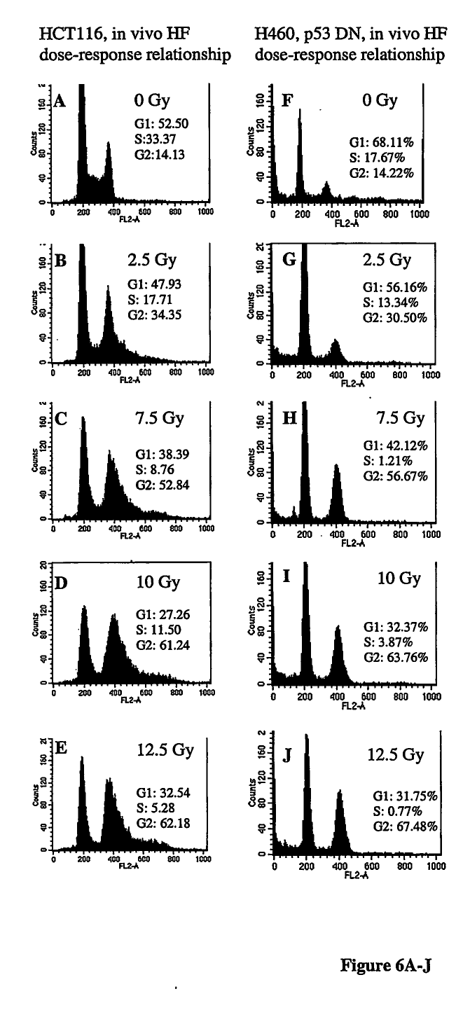

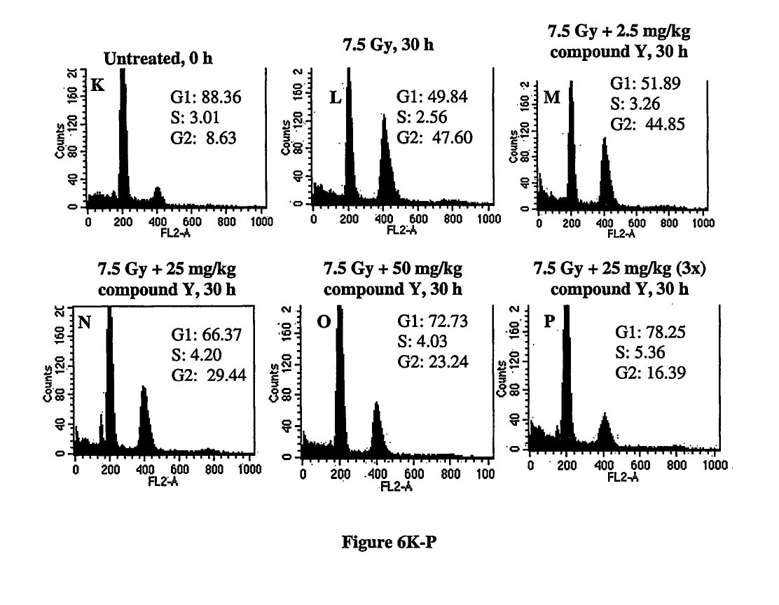

Results show that when topotecan's concentration was raised to 50 nM, a more consistent and robust G2 arrest was observed at 30 hour (about 90% compared to 70% at 30 nM topotecan concentration) after implantation (FIG. 4C, D). Compound Y was shown to be a potent inhibitor of G2 checkpoint in both topotecan (FIG. 4) and gamma irradiation (FIG. 6K-P) PD models (see Table 1B). At 25 mg/kg dose, a single intravenous injection of the compound Y produced significant abrogation of topotecan arrested HCT116 cells as reflected by the parameters:

Odd (the chance for cells to be in G2 phase versus G1 phase, expressed as a ratio between G2% and G1% [G2%/G1%]),

$$Odd Ratio ( OR = 2.63 ) ( OR = \frac{G 2 \% / G 1 \% ( control )}{G 2 \% / G 1 \% ( treatment )} ) and Chi Square analysis ( P = 0.047 ) .$$

At 50 mg/kg, compound Y produced a more pronounced G2 checkpoint inhibition (OR=3.29, p=0.025).

Gamma Irradiation Induced G2 Arrest in a Dose-Dependent Manner in vivo

In vivo dose titration studies with gamma irradiation were completed for two cell lines, HCT116 (p53 WT and isogenic p53 mutant) and H460 (p53 WT and isogenic p53 mutant). Cells in fibres were prepared and implanted, and some mice also received intraperitoneal fiber implantation. A single dose of irradiation at the indicated doses were given to the nude mice carrying the fibres. 24 hours after irradiation, the fibers were retrieved and analyzed by FACS. A dose-dependent G1 and G2 arrest was observed for both cell lines (FIG. 6A-J).

In the treatment groups, nude mice bearing p53 dominant negative H460 cells in the hollow fibers were subject to Gamma Irradiation and compound Y treatment shortly after implantation (time 0 h, FIG. 6K). Compound Y was administered at the indicated doses by either single intravenous (FIGS. 6M, N, O) or 3 intraperitoneal (FIG. 6P) (injected at 0, 8, 24 h) injections to the nude mice bearing the implanted hollow fibers. Hollow fibers were harvested 30 h after implantation and analyzed by FACS analysis.

The results showed that both cell lines arrest at either G1 or G2 or both checkpoints in a dose dependent manner (FIG. 6A-J). For instance, in HCT116 cells, gamma irradiation activates both G1 and G2 checkpoints at the tested doses with G2 arrest more pronounced at higher irradiation doses; more G2 arrest was observed at 7.5, 10 and 12.5 Gy than at 2.5 and 5 Gy. A p53 status-dependent response is also noted for isogenic H460 cell lines. Specifically, in contrast to p53 wild type H460 cells, p53 mutant H460 cells exhibit less G1 arrest but more G2 arrest across the tested dose range (from 2.5 to 12.5 Gy) than wild type H460 cells (FIG. 6, data for the wild type H460 not shown).

Compound Y Inhibits Gamma Irradiation Induced G2 Arrest in vivo

It was further demonstrated that systemic administration of compound Y, either before or after gamma irradiation, inhibited gamma irradiation induced G2 arrest in H460 cells (FIG. 6K, L, M, N, O, P). P values of 0.039, and 0.001 were achieved at single intravenous doses of 25 and 50 mg/kg, respectively, while 3 intraperitoneal injections of compound Y produced a P value of <0.0001 (Table 1-B). Thus, the results show that inhibition of G2 checkpoint by compound Y is more pronounced in the gamma irradiation PD model.

### Example 3

G2 Checkpoint Inhibition by Compound X Correlates with Target Inhibition

The inhibition of G2 arrest by compound X was attributed to the inhibition of chk1 kinase activity as demonstrated by the Western blot analysis of the cells harvested from hollow fibre samples, where in vivo treatment of mice with compound X suppressed phosphorylation of cdc25c at ser 216, the direct substrate for chk1. Samples were collected at various time points from control and treated groups and probed with antibodies specific to total cdc25c (indicated with an arrow), phospho-cdc25c (ser-216, indicated with an arrow), total CDC2 and phospho-CDC2 (tyr-15). The intensity of the bands were further measured with a Fluor-imager (Biorad) and the ratio between phosphorylated and total proteins were plotted. FIG. 5A is a histogram showing phosphor/total cdc25c ratio and FIG. 5B is a histogram showing phosphor/total cdc2 ratio.

The inhibition of phosphorylation at ser 216 resulted in an increase in cdc25c's phosphatase activity and subsequent reduction in phosphorylation of its substrate, CDC2 at tyr 15.

### Example 4

Beyond G2 Checkpoint Regulation

We adapted the study of G2 checkpoint regulation to the study of other cell cycle regulation pathways. Cells were first synchronized at the G1/S boundary by means of a double thymidine block (FIG. 7A-F). After synchronization, cells were harvested at indicated time points for observation of cell cycle progression (7B, C). To arrest cells at G2 checkpoint, the synchronized cells were incubated with topotecan at indicated concentrations for 1 h, and the topotecan was added 30 to 60 minutes after the release of double thymidine block. Topotecan treated cells were then harvested 24 h after double thymidine block and analyzed with FACS. Alternatively, at the end of double thymidine block, fibres were implanted to mice subcutaneously (4 fibres per mouse); fibres were retrieved from mice at the specified time points (8 fibres per time point) and analyzed by FACS. Control fibers without double thymidine treatment (8A, F) were also included.

The results showed, importantly, that double thymidine block also effectively synchronizes/arrests cells loaded in hollow fibres (FIG. 8). Upon release of the block, the synchronized cells sequentially progress through S and G2/M phases and reenter G1 phase over a period of about 24 hours (FIGS. 7, 8). Additionally, we showed that double thymidine block synchronized cells also arrest at the G2 checkpoint with brief topotecan treatment in a dose dependent manner (FIGS. 7 D, E, F). The G1-arrested cells were exposed to 1 h topotecan treatment; and the cells were arrested at G2 checkpoint 24 h after removal of double thymidine block. 2 μM of Topotecan achieves complete G2 arrest compared to 100 nM while 5 μM topotecan starts to have noticeable S phase arrest as well. Therefore, by synchronizing cells at G1/S boundary and eliciting a G2 arrest with a brief topotecan treatment, this modified approach allows the study of molecular targets such as CDKs 1, 2 and 4 as well as the regulatory pathways including S and G2 checkpoint pathways interfacing with them.

### Example 5

PD Activity and Bone Marrow Toxicity

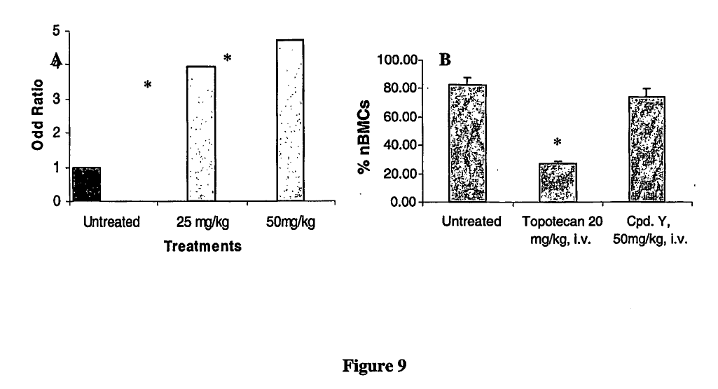

PD activity and bone marrow toxicity for compound Y were examined in mice. Thirty hours after fiber implantation and compound treatment, hollow fibers and/or bone marrow were harvested from the same mice received either no treatment, Topotecan (single intravenous bolus injection at 20 mg/kg) or compound Y (25 and 50 mg/kg) by intravenous injection (FIG. 9A). The inhibition of G2 checkpoint by compound Y is expressed as an increase in odd ratio values in the treatment groups. A dose dependent G2 checkpoint inhibition is observed for compound Y (FIG. 9A). The percentage of nucleated bone marrow cells was used as an indicator of bone marrow toxicity. Topotecan, an agent know to cause bone marrow suppression, was included as a positive control. No statistically significant bone marrow toxicity was observed for compound Y at the tested dose (FIG. 9B). * indicates P<0.05.

### Example 6

Simultaneous Study of PD, PK and Bone Marrow Toxicity in Rats.

A simultaneous study of PD, PK and bone marrow toxicity with compound Y in rats was performed. The rats were implanted with the hollow fibres containing Topotecan (30 nM) arrested HCT116 cells at time 0 h. Compound Y was dosed by a single intravenous (iv) injection at 1.0, 2.5, 5.0, 10 mg/kg, respectively, immediately after the implantation (0 h). 30 h after implantation, hollow fibres and bone marrows were harvested simultaneously and analyzed by FACS. In addition, plasma samples were collected at the indicated time points via a catheter implanted to the jugular vein of the rats. Bone marrow cells were stained with LDS-751 (a marker for nucleated bone marrow cells) prior to FACS analysis. Hollow fibres were also taken from the same rats collected for plasma at the same time points and the concentration of the compound Y in the hollow fibres determined. A dose-dependent inhibition of the G2 checkpoint by compound Y is shown in (FIG. 10A). The bone marrow analysis result is shown in (FIG. 10B). Pharmacokinetics of the compound Y at the indicated doses and time points is shown in (FIG. 10C). The pharmacokinetics for compound Y in hollow fibres and plasma collected from the same rats at 5 mg/kg are shown in (FIG. 10D). * indicates P<0.05.

We demonstrate here that the PD model can also be used in conjunction with PK and toxicity evaluation in either mice or rats to study the PK-PD relationship and evaluate the therapeutic margin of G2 checkpoint inhibitors. In addition, the concentration of the compound in the hollow fibres can also be measured directly and correlated to the plasma pharmacokinetics collected from the same rats (FIG. 10). The bone marrow toxicity was measured by following the percentage of nucleated bone marrow cells in the bone marrow after compound treatment.

In mice, compound Y inhibited G2 checkpoint at 25 and 50 mg/kg without causing bone marrow toxicity (FIG. 9). In rats, compound Y exhibited a dose-dependent G2 checkpoint inhibition from 1 to 10 mg/kg and the bone marrows harvested from the same rats did not show statistically significant bone marrow toxicity up to 10 mg/kg after single iv injections (FIG. 10). A significant inhibition of G2 checkpoint was observed at a low dose of 2.5 mg/kg (P=0.05) (FIG. 10). The observed PD activity was then directly correlated to the PK profiles of compound Y at the indicated doses (FIG. 10A, C). Importantly, the concentrations of the compound Y in the hollow fibres closely follow that of the plasma after equilibrium is established 2 hours after compound Y administration. Collectively, these parameters indicate a therapeutic margin (the ratio between the toxic dose (>10 mg/kg) and the efficacious dose (2.5 mg/kg) of 4 or higher for compound Y.

The PK and PD results derived from these studies enables us to define the relationship between compound exposure time in the plasma or hollow fiber and pharmacologic activity of the compound. The definition of the PK-PD relationship also provides the basis for dose selection in xenograft efficacy study design.

### Example 7

Potential Applications of the Pharmacodynamic (PD) Method in Drug Discovery

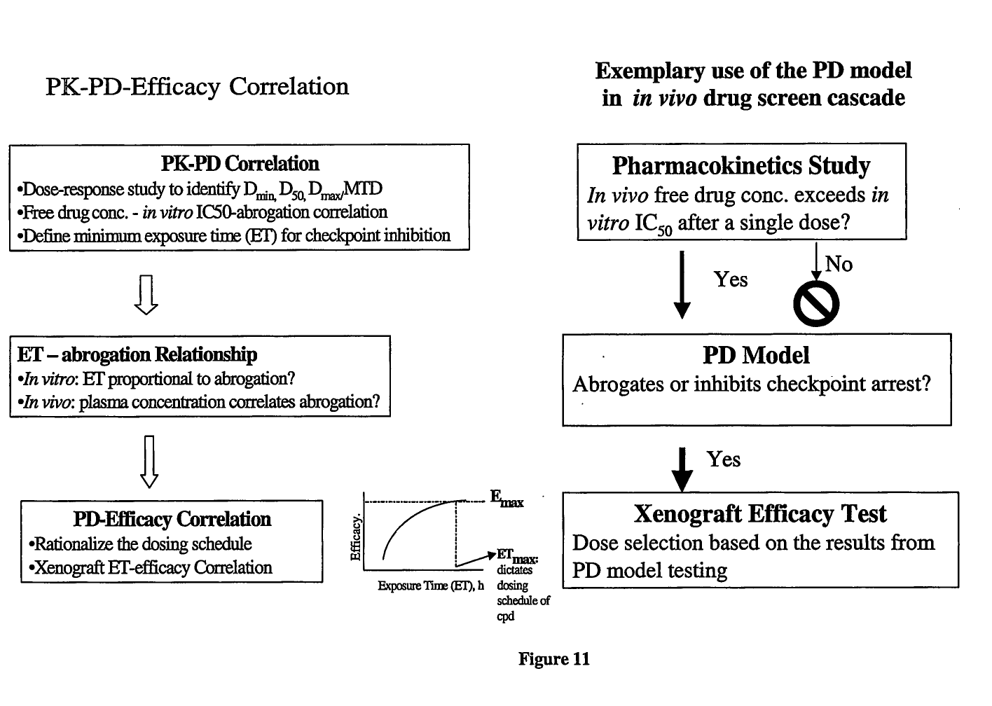

FIG. 11 demonstrates the potential applications of the pharmacodynamic (PD) method in drug discovery. The PD method is useful in establishing PK-PD-Efficacy relationship, which is essential for screening compounds, identifying optimal biological doses and designing dosing regimen for preclinical as well as clinical efficacy test. For instance, with a given G2 checkpoint pathway inhibitor such as compound X, the PD method not only allows the establishment of dose-response relationship, but also enables the study of exposure time (the duration of time that the drug concentration is maintained above the minimum effective concentration such as EC50 or IC50 determined by in vitro cell based assay) and checkpoint inhibition relationship, which should guide the dosing regimen selection for further efficacy tests. Additionally, the PD method can be used in the drug screen cascade where quantitative criteria can be set, and unknown compounds will be compared to the reference compound at the desired doses for in vivo pharmacodynamic activity. If the set criteria are met, the compound can be used for further efficacy testing. Abbreviations: ET (exposure time); MTD (maximum tolerated dose); PK (pharmacokinetics), Dₘᵢₙ (minimum effective dose), D₅₀ (the dose that produces 50% of desired activity), ETₘₐₓ (exposure time corresponding to maximum efficacy), Eₘₐₓ (maximum efficacy), cpd (compound), IC₅₀ (the concentration of a compound that produces 50% inhibition of the target protein activity), IR (irradiation).

### Example 5

Quantifiable Parameters for the Pharmacodynamic Model

A quantifiable set of parameters can be used to ascertain both the quality of the experiment as well as the extent of cell cycle checkpoint inhibition. The potential parameters that can be used to quantitate the extent of G2 checkpoint inhibition are summarized in Table 1 and listed below:
1) Spontaneous abrogation in control (SΔ % G2)=% G2 at 24 or 30 h-% G2 at 0 h.
2) Net change in % G1 compared to control (Δ % G1) at 24 or 30 h.
3) Net change in % G2 compared to control (Δ % G2) at 24 or 30 h.
4) Combined change in % G1 and % G2 (Δ % G1+Δ % G2).
5) The ratio between % G2 and % G1 (% G2/% G1) at 0, 24 or 30 h, also called Odd. In addition, Odd Ratio (OR) is calculated by dividing Odd (control) by Odd (treatment):

$$( OR = \frac{G 2 \% / G 1 \% ( control )}{G 2 \% / G 1 \% ( treatment )} ) .$$
6) The ratio between Δ % G2 in treatment group and % G2 in the control group (Δ % G2/% G2.control) at 24 or30 h.

Note: Depending on the cell type and DNA damaging method, a range of time points from a few hours to a few days can be used in addition to the 24 and 30 h time points.

Among the six parameters, Δ % G2 is a direct reflection of the absolute reduction in % G2 cells as a result of checkpoint inhibition. Δ % G2 is useful in comparing the in vivo checkpoint inhibition potency of compounds within the same experiment.

By taking into account the variation of the control group, Δ % G2/% G2.control is a useful parameter for comparing in vivo potency across the experiments since it examines the relative change between the treatment and the control group within the same experiment.

% G2/% G1 (also called ‘odd’) appears to be the most sensitive indicator for checkpoint inhibition, which in addition serves as a parameter indicating the quality (extent of G2 arrest) of the control group. For instance, the control groups had % G2/% G1 values greater than 3.0, while compound X treated groups showed values ranging from 0.75 to 1.35. Alternatively, the inhibition activity of a test compound can be expressed as odds ratio (the ratio between the control % G2/% G1 and treatment group % G2/% G1). Conceivably, odd and odd ratio can be used to quantitate and rank the activity of checkpoint pathway inhibitors.

Alternatively, SΔ % G2 reflects the spontaneous G2 checkpoint inhibition in the control group (topotecan alone).

The other two parameters (Δ % G1, Δ % G1+Δ % G2) provide essentially the same information as Δ % G2 when cells reenter G1 cell cycle after exiting G2 arrest.

To summarize, SΔ % G2 and % G2/% G1, and Odd ratio are potentially useful parameters reflecting the quality and validity of the experiment, while % G2/% G1 in addition serves as a sensitive quantitative indicator of checkpoint inhibition activity. Altematively, Δ % G2/% G2.control can be a useful parameter enabling comparison of different compounds across experiments.

TABLE 1-A
summary of parameters for in vivo pharmacodynamic method

| | | | | | | | | | | Δ% G2/ |
|---|---|---|---|---|---|---|---|---|---|---|
| | | 0 h | 24 h | | SΔ | Δ% | | Δ% G1 + Δ% | Δ% G2/ | Δ% G2. |
| Doses | Study | G2 % | % G1 | % G2 | % G2 | G2 | Δ% G1 | G2 | Δ% G1 | control |
| Control | Exp. 1 | 83.00 | 23.00 | 71.00 | −12.00 | | | | 3.09 | |
| TOP | | | | | | | | | | |
| Caffeine, | Exp. 1 | | 41.00 | 50.00 | | 21.00 | 18.00 | 39.00 | 1.22 | 0.30 |
| 150 mg/kg | | | | | | | | | | |
| Control | Exp. 2 | 69.00 | 16.00 | 58.00 | −11.00 | | | | 3.63 | |
| TOP | | | | | | | | | | |
| Compd X, | Exp. 2 | | 38.00 | 36.00 | | 22.00 | 22.00 | 44.00 | 0.95 | 0.38 |
| 50 mg/kg | | | | | | | | | | |
| Compd X, | Exp. 2 | | 32.00 | 42.00 | | 16.00 | 16.00 | 32.00 | 1.31 | 0.28 |
| 25 mg/kg | | | | | | | | | | |
| Caffeine, | Exp. 2 | | 11.00 | 51.00 | | 7.00 | −4.00 | 3.00 | 4.64 | 0.12 |
| 150 mg/kg | | | | | | | | | | |

TABLE 1-B
Quantitative evaluation of G2 checkpoint inhibition by compound Y in topotecan and
gamma irradiation pharmacodynamic models. n = 2 or 3.

| Treatments | % G1 | % G2 | Odd (% G2/% G1) | Odd ratio | CI (95%) | P value |
|---|---|---|---|---|---|---|
| TOP alone | 7.3 | 88.1 | 12.0 | | | |
| TOP alone | 7.2 | 90.2 | 12.5 | | | |
| TOP alone | 9.0 | 87.9 | 9.7 | | | |
| Mean | 7.9 | 88.7 | 11.4 | 1.0 | | |
| Compound Y, 25 mg/kg, 1x, iv | 17.7 | 68.8 | 3.9 | | | |
| Compound Y, 25 mg/kg, 1x, iv | 15.8 | 71.4 | 4.5 | | | |
| Mean | 16.7 | 70.1 | 4.2 | 2.63 | 1-5.99 | 0.047 |
| Compound Y, 50 mg/kg, 1x, iv | 18.4 | 71.7 | 3.9 | | | |
| Compound Y, 50 mg/kg, 1x, iv | 19.8 | 66.6 | 3.4 | | | |
| Compound Y, 50 mg/kg, 1x, iv | 23.3 | 66.8 | 2.9 | | | |
| Mean | 20.5 | 68.4 | 3.4 | 3.29 | 1.1-6.4 | 0.025 |
| Un-irradiated control | 86.9 | 3.6 | 0.04 | | | |
| Un-irradiated control | 86.6 | 2.8 | 0.03 | | | |
| Un-irradiated control | 88.4 | 8.6 | 0.10 | | | |
| Mean | 87.3 | 5.0 | 0.06 | | | |
| IR, 7.5 Gy | 54.2 | 42.2 | 0.78 | | | |
| IR, 7.5 Gy | 55.1 | 42.3 | 0.77 | | | |
| IR, 7.5 Gy | 49.8 | 47.6 | 0.96 | | | |
| Mean | 53.0 | 44.0 | 0.83 | 1.00 | | |
| Compound Y, 2.5 mg/kg, 1x, iv | 51.1 | 47.1 | 0.92 | | | |
| Compound Y, 2.5 mg/kg, 1x, iv | 51.9 | 44.9 | 0.87 | | | |
| Compound Y, 2.5 mg/kg, 1x, iv | 51.2 | 44.9 | 0.88 | | | |
| Mean | 51.4 | 45.6 | 0.89 | 0.93 | 0.53-1.65 | 0.820 |
| Compound Y, 25 mg/kg, 1x, iv | 61.8 | 35.2 | 0.57 | | | |
| Compound Y, 25 mg/kg, 1x, iv | 70.7 | 24.5 | 0.35 | | | |
| Compound Y, 25 mg/kg, 1x, iv | 66.4 | 29.4 | 0.44 | | | |
| Mean | 66.3 | 29.7 | 0.45 | 1.83 | 1.03-3.34 | 0.039 |
| Compound Y, 50 mg/kg, 1x, iv | 77.6 | 17.5 | 0.23 | | | |
| Compound Y, 50 mg/kg, 1x, iv | 70.9 | 24.8 | 0.35 | | | |
| Compound Y, 50 mg/kg, 1x, iv | 72.7 | 23.2 | 0.32 | | | |
| Mean | 73.7 | 21.8 | 0.30 | 2.78 | 1.51-5.23 | 0.001 |
| Compound Y, 25 mg/kg, ip, 3x | 78.3 | 16.4 | 0.21 | | | |
| Compound Y, 25 mg/kg, ip, 3x | 83.3 | 7.7 | 0.09 | | | |
| Compound Y, 25 mg/kg, ip, 3x | 75.2 | 13.0 | 0.17 | | | |
| Mean | 78.9 | 12.4 | 0.16 | 5.24 | 2.58-10.89 | 0.000 |

Materials and Methods
Cells

The H460 and HCT116 cell lines were acquired from American Type Culture Collection, Manassas, Va. H460 cells were maintained in RPMI media supplemented with 10% fetal bovine serum. HCT116 cells were maintained in McCoy's 5a medium supplemented with 10% fetal bovine serum. p53 dominant negative cell lines were generated by stable transfection of cells with the retroviral vector pLXSN expressing the tetramerization domain of p53.

Animals

Male athymic NCr nu/nu mice at 6-8 weeks of age purchased from Taconic Farm (Germantown, N.J.), and Sprague-Dawley rats purchased from Charles River Laboratories (Wilmington, Mass.) were used in this study. All procedures were performed according to a research protocol approved by Animal Care and Use Committee at AstraZeneca R&D Boston. The animals were housed 5 per cage in sterile, polycarbonate, filter-capped micro-isolation cages. All animals were maintained in a barrier facility on 12 hour light/dark cycles and provided food and water ad libitum.

Chemicals

Topotecan (TOP) was purchased from C.Q international Co. Inc. (Cambridge, Mass.). Topotecan was dissolved in saline and prepared fresh for each use. Caffeine was dissolved in saline to a concentration of 20 mg/mL, filtered, and injected intraperitoneally to mice right before and 5 h after fibre implantation. Go6976 was purchased from Calbiochem (San Diego, Calif.) as DMSO solution at 0.5 mg/mL. Compounds X and Y are selective inhibitors of chk1 kinase. LDS-751 was purchased from Molecular Probes/Invitrogen (Carlsbad, Calif.).

Hollow Fibre Preparation

The procedures were performed essentially as described (Hall, L. A. et al. Anticancer Res 20, 903-11 (2000); Hollingshead, M. G. et al. Life Sci 57, 131-4 (1995)). Polyvinylidene fluoride (PVDF) hollow fibres (500,000 Da molecular weight cut-off, 1.0 mm ID) were purchased from Spectrum Medical Industries, Houston, Tex. The fibres were individually flushed and filled with 70% ethanol and incubated in 70% ethanol at room temperature for a minimum of 96 hours. Following 3 washes with deionized water, the fibres were filled with water and placed into a pan of deionized water for sterilization by autoclaving. Hollow fibres are washed with either RPMI or McCoy's medium containing 20% FBS prior to loading with cells.

Fibre Loading and Implantation

The procedures were performed essentially as described (Hall, L. A. et al. Anticancer Res 20, 903-11 (2000); Hollingshead, M. G. et al. Life Sci 57, 131-4 (1995)). Briefly, prior to filling with cells, each fibre was flushed with cold medium appropriate to the cell line. Cells at log growth phase were harvested for hollow fibre loading. The cell suspension was put into 5 cc syringe and filled into each fibre. The loading concentration of cells was optimized to 3×10⁶ per mL. The ends of fibres were heat-sealed. Single implants were formed by crimping the fibre at 2 to 5 cm sections and applying a heat-seal at the crimp sites. Implants were then transferred into culture dishes containing complete medium, incubated at 37° C. overnight in 5% CO2 prior to further manipulation.

Extracting Cells from Hollow Fibre

At indicated time points, fibres were removed from mice, wiped with gauze and placed in petri dishes or 6 well plates containing enough warm media to cover fibres. Both ends of the fibres were then cut open using forceps and scissors. The fibres were flushed twice with a 200 μL pipette filled with medium by inserting into fibre opening. The medium containing the cells were collected and centrifuged for 3-minutes at 1600 rpm. The supernatant was discarded and 1 mL of trypsin was added onto the pellets and mixed until single cell suspension is obtained. Then, 5 mL of serum rich medium was added to the cell suspension and centrifuged. The supernatant was then decanted leaving approximately 200 μL of cell suspension. The suspension was then added. drop wise while vortexing to 5 mL of ice cold 70% ETOH. Cells were then fixed at −20° C. for a minimum of 1 hour. The cells obtained were then processed according to FACS staining protocol. 3-8 fibres from each time point were typically used for FACS analysis.

Double Thymidine Block and G2 Arrest with Brief Topotecan Treatment

Cells were plated onto 10-cm dishes on day 1 such that 30% confluence is reached on day 2. In hollow fibre experiments, loaded fibres were made on day 1, and incubated in the dishes overnight. On day 2, thymidine was added to the dishes to a final concentration of 2 mM. After 16 hours of incubation, dishes were washed 3 times with PBS, refilled with fresh medium, and incubated for 10 hours. Then, the second thymidine treatment was applied for another 14 hours. At the end of double thymidine block, dishes were washed three times with PBS, refilled with complete medium, and incubated for 30 minutes in the incubator prior to addition of topotecan at concentrations of 0, 0.1, 2.0, and 5.0 μM. The topotecan was washed away after 1 hour treatment, and the dishes were refilled with fresh medium. Cells were harvested 24 hours after the second thymidine treatment, and analyzed by FACS analysis. Additional samples including untreated controls were harvested at indicated time points to follow cell cycle progression.

G2 Arrest with Prolonged Topotecan Treatment

Cells were plated in 10 cm dishes on day 1 such that 30-50% confluence is reached on day 2. On day 2, topotecan was added to the dishes at concentrations of 0, 5, 10, 25, 50, and 100 nM and incubated for 18 hours in 37° C. incubators. At the end of incubation, dishes were washed 3 times with PBS and then refilled with fresh medium. Cells were sampled at 0, 6, 9, 24 and 30 hours for FACS analysis. For hollow fibre experiment, cell-loaded fibres were prepared on day 1 as described and treated with topotecan as described for cells on day 2. Fibres were implanted to mice at the end of 18-hour topotecan treatment. Some fibres were harvested from mice at 0, 9 24 and 30 h and analyzed by FACS.

In vivo Inhibition

Two compounds were tested in this PD model using either topotecan or gamma irradiation as DNA damaging agents. Caffeine was dosed to animals at 150 mg/kg and Compound X was dosed at 25 and 50 mg/kg per dose. A total of two intraperitoneal doses were given with the first dose administered immediately after fibre implantation (topotecan experiment) (0 h) or before gamma irradiation and the second dose 5 h later. Fibres were harvested 24 h after implantation and examined by FACS and western blot analysis. In addition, fibres at time 0 h (time of implantation) were also harvested for FACS and western blot studies.

FACS Analysis

Fixed cells were centrifuged at 1600 rpm for 5 minutes and the ETOH was decanted. They were then washed with 2 ml of PBS, recentrifuged and decanted. The cells were then stained with propidium iodide. Cell cycle study was performed using a FACSCalibur (Becton Dickinson) using Cell-QuestPro software. Cell cycle distribution was determined using either Modfit (Verity software house, ME, USA) or WinMDI (Joseph Trotter, Scripps Institute) software.

Western Blot

The cells were harvested in cold modified RIPA buffer (0.15 M NaCl, 0.05 M Tris-HCl, pH 7.4, 1% Triton-100, 1% sodium deoxycholate) containing enzyme inhibitors. The protein concentration of the cell lysates were measured with micro-BCA reagent kit (Pierce, Ill.) and adjusted to 0.45 mg/mL for SDS-PAGE electrophoresis. 12 μg of proteins were loaded onto 4-12% Bis-Tris gel (invitrogen, WI), electrophoresed for 50 minutes at 200 voltage, transferred to nitrocellulose membranes and probed with cdc25c and CDC2 specific antibodies.

Gamma Irradiation

Hollow fibres were prepared as described on day 1, and incubated overnight. On day 2, fibres were implanted to mice as described above. On day 3, mice carrying fibres were irradiated at the indicated doses with a Cs-137 Gamma Irradiator (MARK I, JL Shepherd & Associates, CA). Fibres were harvested either 24 or 30 h after irradiation and analyzed by either FACS or western blot. Compounds were administered to mice either before or after Gamma Irradiation according to the design of the experiment.

Bone Marrow Toxicity Model

Femurs harvested from mouse or rat were tried off muscular tissue, and the femoral head and distal epiphysis of femur were cut off. Bone marrow tissue was flushed 5 times with 2 mL of PBS containing 50% FBS using a 19-gauge needle. A single cell suspension was: made by passing the suspension through the needle a few times and filtering through a 100 μM disposable filter. The cell suspension was then centrifuged, aspirated and re-suspended in 4 mL of ice cold PBS containing 0.5% BSA. The concentration of the cells was adjusted to 2.5×10⁶ per mL. 400 μL of the adjusted suspension was added with 20 μL of LDS-751 staining solution, incubated for 20 minutes in the dark at room temperature, and analyzed by FACS. The ratio between nucleated bone marrow cells (WBCs) and un-nucleated red blood cells (RBCs) were used to monitor the bone marrow toxicity of compounds.

PK-PD-Tox Study with Compound Y in Rats

Fibres loaded with HCT 116 cells were prepared on day 1 as described and treated with either 30 or 50 nM of topotecan for 18 hour on day 2. Fibres were implanted to the cannulated rats at the end of 18-hour topotecan treatment. Some fibres were harvested from the control animals at 0, and 30 h and analyzed by FACS. In the treatment group, compound Y was administered at indicated doses by a single intravenous injection immediately after the fibre implantation. Plasma samples and hollow fibres were collected at the indicated time points and analyzed for pharmacokinetics. 30 h after implantation, both fibres and the bone marrow samples were harvested and analyzed by FACS to determine the G2 checkpoint inhibition and bone marrow toxicity caused by compound Y.

Measurement of Compound Concentration in Hollow Fibres

At time points indicated rats are anesthetized using isofluorane. The wound clip is removed and using sterile forceps two 5 cm fibers are removed. The wound is then re-closed using a new wound clip. Then, the fibers are wiped clean with gauze and both heat sealed ends are cut off. One end of the fiber is placed into an eppendorf tube while a pipette tip is placed into the other end and the fibers are flushed with air. The samples are then frozen at −20° C. until analysis.

1. An in vivo pharmacodynamic method for testing a compound for cell cycle regulation comprising:
i) implanting a semi-permeable cell receptacle comprising a cell into an animal;
ii) administering a test compound to said animal in vivo; and
iii) determining a cell cycle endpoint in the cell, whereby a progression or arrest of a cell cycle phase in the cell indicates that the compound is a cell cycle regulator.

2. The method according to claim 1 in which the semi-permeable cell receptacle is a hollow fibre.

3. The method according to claim 1 in which FACS analysis is used to measure the cell cycle endpoint.

4. The method according to claim 1 in which the cells are arrested at the G1 phase.

5. The method according to claim 1 in which the cells are arrested at the G2 phase.

6. The method according to claim 1 in which the cells are arrested at the G1 phase and the G2 phase.

7. The method according to any of claims 4-6 in which the cells are arrested at the G1 and/or G2 phase by administering a DNA damaging agent, an antimetabolite and/or an antiproliferative.

8. The method according to claim 7 in which the DNA damaging agent is topotecan.

9. The method according to claim 7 in which the DNA damaging agent is gamma irradiation.

10. The method according to claim 7 in which the antiproliferative is a thymidine block.

11. The method according to any preceding claim further comprising the step of determining whether a protein associated with the cell cycle phase is affected by the compound.

12. The method according to claim 11 in which determining whether the protein associated with the cell cycle phase is affected by the compound comprise:
i) lysing the cells to produce a cell extract containing proteins from the cell;
ii) separating proteins from the cell extract to produce a profile of proteins; and
iii) comparing the profile to a profile obtained from cells not so treated with the compound.

13. The method according to claim 12 in which Western blot analysis is used to produce the profiles that are compared.

14. The method according to any one of claims 11-13 in which the protein is associated with the G1 phase, S phase, G2 phase or the M phase.

15. The method according to any one of claims 11-14 in which the proteins are associated with the G2 checkpoint pathway, the proteins being selected from the group consisting of p21, p53, ATR, ATM, Chk1, Chk2, CDK1 (CDC2), Myt 1, Wee 1, Cdc25c, Cdc25A and cyclin B.

16. The method according to any of claims 11-15 in which the protein is cdc25c.

17. The method according to any one of claims 11-15 in which the protein is CDK1 (CDC2).

18. The method according to claim 1 in which the cells are peripheral blood mononuclear cells.

19. The method according to claim 1 in which the cells are tumour cells.

20. The method according to claim 19 in which the tumour cells are HCT116 cells or H460 cells.

21. The method according to claim 1 in which compound is administered systemically to the animal.

22. The method according to claim 1 in which the animal is selected from the group consisting of a human, rodent, rabbit, dog, rhesus monkey and chimpanzee.

23. The method according to claim 1 in which the animal is a rodent.

24. The method according to claim 23 in which the rodent is a rat.

25. The method according to claim 23 in which the rodent is a mouse.

26. Use of a method as defined in any preceding claim for performing a pharmacokinetic-pharmacodynamic-efficacy correlation study.

27. Use of a method according to claim 26 to determine an optimal biological dose of compound for an in vivo efficacy study.

28. Use of the optimum biological dose determined according to claim 27 in a clinical trial design protocol.

29. Use of the optimum biological dose according to claim 28 in a human clinical trial design protocol.
